(19) United States
(12) Patent Application Publication
(10) Pub. No.: US 2023/0220322 A1
(43) Pub. Date: Jul. 13, 2023

(54) BIOCOMPATIBLE DEVICE WITH AN ADSORBED LAYER OF CATIONIC COMB COPOLYMER

(71) Applicant: faCellitate GmbH, Mannheim (DE)

(72) Inventors: Tobias MENTZEL, Limburgerhof (DE); Nadine KAISER, Ludwigshafen Am Rhein (DE); Veronique SCHWARTZ, Mannheim (DE); Rupert KONRADI, Ludwigshafen Am Rhein (DE); Peter HAHN, Ludwigshafen Am Rhein (DE); Peter STENGEL, Ludwigshafen Am Rhein (DE); Carina OBERFRANK, Ludwigshafen Am Rhein (DE)

(21) Appl. No.: 18/011,056

(22) PCT Filed: Jun. 8, 2021
(86) PCT No.: PCT/EP2021/065281

§ 371 Date: Dec. 16, 2022

(30) Foreign Application Priority Data
Jun. 19, 2020 (EP) 20181193.2

(51) Int. Cl. C12M 1/12; C08L 33/26

(52) U.S. Cl. CPC C12M 25/14; C08L 33/26; C08L 2203/02

(57) ABSTRACT

The present invention relates to a biocompatible device which comprises on its surface an adsorbed layer of a polymer P which is a copolymer of at least one macromonomer selected from an ester E of (meth)acrylic acid and polyethylene oxide or a polyethylene glycol (meth)acrylamide, at least one monomer M selected from alkyl (meth)acrylate, aryloxyalkyl (meth)acrylate, alkyl (meth)acrylamide or aryl (meth)acrylamide, and at least one cationic monomer C selected from cationic ethylenically unsaturated N-containing monomers. It further relates to a process for making a biocompatible device which comprises on its surface an adsorbed layer of the polymer P comprising the following steps: providing a biocompatible device, and applying to the surface of the biocompatible device a solution S of the polymer Pin a solvent L. It further relates to a solution S comprising the polymer P in the solvent L, where the solvent L comprises an alcohol; and to a process for cultivating cells, comprising the following steps: providing the biocompatible device and cultivating the cells in the supernatant medium above the surface of the biocompatible device.

The present invention relates to a biocompatible device which comprises on its surface an adsorbed layer of a polymer P which is a copolymer of at least one macromonomer selected from an ester E of (meth)acrylic acid and polyethylene oxide or a polyethylene glycol (meth)acrylamide, at least one monomer M selected from alkyl (meth)acrylate, aryloxyalkyl (meth)acrylate, alkyl (meth)acrylamide or aryl (meth)acrylamide, and at least one cationic monomer C selected from cationic ethylenically unsaturated N-containing monomers. It further relates to a process for making a biocompatible device which comprises on its surface an adsorbed layer of the polymer P comprising the following steps: providing a biocompatible device, and applying to the surface of the biocompatible device a solution S of the polymer P in a solvent L. It further relates to a solution S comprising the polymer P in the solvent L, where the solvent L comprises an alcohol; and to a process for cultivating cells, comprising the following steps: providing the biocompatible device and cultivating the cells in the supernatant medium above the surface of the biocompatible device. Combinations of preferred embodiments with other preferred embodiments are within the scope of the present invention.

Biocompatible devices such as biosensors and devices for cultivation of cells play an important role in many technologies. An important issue with the application of biocompatible devices is the unwanted deposition of biological or organic material on a surface. For example, when cultivating cells, some types of cells tend to attach to the surface or proteins may be deposited. For biosensors, one undesired side effect is that the signal-to-noise ratio of the detected signals and the limit of detection (LOD) is worsened. Several approaches have been tried to solve these problems and to prevent the formation and deposition of organic or biological materials on a surface of biocompatible devices.

This objective was achieved by a biocompatible device which comprises on its surface an adsorbed layer of a polymer P which is a copolymer of
at least one macromonomer selected from an ester E of (meth)acrylic acid and polyethylene oxide or a polyethylene glycol (meth)acrylamide,
at least one monomer M selected from alkyl (meth)acrylate, aryloxyalkyl (meth)acrylate, alkyl (meth)acrylamide or aryl (meth)acrylamide, and
at least one cationic monomer C selected from cationic ethylenically unsaturated N-containing monomers.

The object was also achieved by a process for making a biocompatible device which comprises on its surface an adsorbed layer of a polymer P which is a copolymer of
at least one macromonomer selected from an ester E of (meth)acrylic acid and polyethylene oxide or a polyethylene glycol (meth)acrylamide,
at least one monomer M selected from alkyl (meth)acrylate, aryloxyalkyl (meth)acrylate, alkyl (meth)acrylamide or aryl (meth)acrylamide, and
at least one cationic monomer C selected from cationic ethylenically unsaturated N-containing monomers,
comprising the following steps:
A) providing a biocompatible device, and
B) applying to the surface of the biocompatible device a solution S of the polymer P in a solvent L.

The object was also achieved a solution S comprising the polymer P in the solvent L, where the solvent L comprises an alcohol, such as methanol, ethanol, n/iso-propanol or n/sec/iso/tert-butanol.

The object was also achieved by a process for cultivating cells, comprising the following steps:
a) providing the biocompatible device which is the device for cultivating cells or obtainable by the process for making the biocompatible device, and
b) cultivating the cells in the supernatant medium above the surface of the biocompatible device.

A suitable biocompatible device is a biosensor or a device for cultivating cells.

A device for cultivating cells can for example be any device that is suitable for cultivating cells or for handling such cell. In another form devices can for example be any device that is suitable for cultivating cells including handling such cell. A reference herein to a particular “cell culture” device is understood also to mean corresponding tissue culture devices, for example tissue culture vessels.

Examples of devices for cultivating cells include cell culture flasks, cell culture dishes, cell culture plates (e.g. multiwell or microwell plates), cell culture bags, roller bottles and reactors (e.g. bioreactors).

Further examples of devices for cultivating cells which are suitable for handling cell culture containing liquids include tubes, probes, vials, bottles, pipettes and pipette tips, syringes, microscopic slides, microfluidic devices, glass capillaries, biochips, SiO₂ wavers or SiO₂ arrays.

In a preferred form the device for cultivating cells is selected from cell culture flasks, cell culture dishes, cell culture plates, cell culture bags, reactors, tubes, probes, vials, bottles, pipettes, pipette tips, syringes, microscopic slides, microfluidic devices, glass capillaries, biochips, SiO₂ wavers or SiO₂ arrays.

In another preferred form, the device for cultivating cells is selected from cell culture flasks, cell culture dishes, bioreactors, test tubes, vials, bottles, pipettes, syringes, microscopic slides, microfluidic devices, glass capillaries, biochips, SiO₂ wavers, or SiO₂ arrays.

The devices for cultivating cells can be lab scale devices, semi industrial size devices or industrial size devices. Typical industrial sizes of reactors, such as bioreactors are for instance 3 L, 50-200 L or 1000-2000 L. Typical sizes of a well in a cell culture plate is from 0.001 to 10 ml.

A biosensor is usually an analytical device designed to detect an analyte. The biosensors normally comprise at least one bio-recognition element, a biotransducer component and in many cases an electronic processing system. In many cases the biosensors also comprise tubes or pipes that are designed to transport fluids comprising the analyte towards and away from the biorecognition element as well as a housing to protect the biosensor. An analyte may mean a substance that shall be detected in an analytical process. Normally bioreceptors are biomolecules from organisms or receptors modeled after biological systems to interact with the analyte of interest. Typical bioreceptors include proteins, antibody/antigen, enzymes, nucleic acids/DNA, sugars, carbohydrates, cells or cellular structures, or biomimetic materials. In a preferred embodiment, bioreceptors are proteins such as antigens and antibodies, peptides, DNA, RNA, sugars and carbohydrates.

The biocompatible device comprises on its surface an adsorbed layer of the polymer P. The whole surface or parts of the surface comprises the adsorbed layer of the polymer P. Preferably, at least the part of the surface which usually comes into contact with biological material (e.g. cells) under normal operating conditions comprises the adsorbed layer of the polymer P. The layer of the polymer P is adsorbed to the surface, which usually means that there are no covalent chemical bonds between the polymer and the surface. The term “adsorbed” usually refers to physisorption, and usually not to chemisorption.

Preferably, the surface is free of other adsorbed polymers beside the polymer P. Preferably, the surface is free of other layers (e.g. adsorbed, or covalently bound layers) beside the adsorbed layer of the polymer P.

The adsorbed layer may comprise at least one polymer P, such as one, two or three different polymers P. Preferably, the adsorbed layer consists of the polymer P. In one form the adsorbed layer is free of other polymers beside the polymer P. In another form the adsorbed layer is free of biological compounds, pharmaceuticals or biologically active compounds.

The surface of biocompatible device can be made of any biocompatible material, e.g. material on which cells can be grown, be it with the cells being attached to the surface or not. For example the surface is at least partly made of glass, quartz, silicon, metals, metal oxides or organic polymers, preferably glass. Suitable glass can be fused quartz glass (also called fuse silica), soda-lime glass, borosilicate glass, lead glass, aluminosilicate glass, glass-ceramics and fiberglass. Preferred material is fused quartz glass and soda-lime glass.

In another form, the surface is at least partly made of organic polymers, such as polycarbonate, polystyrene, hydrophilized polystyrene, polyamide, poly(methyl methacrylate), polyesters, polysulfones (like polyethersulfones), polyvinylchloride, polyvinylidene chloride, fluorinated or partially fluorinated polyolefins (like fluorinated polyethylene or polypropylene), polyolefines [such as polyethylene (like low density polyethylene, ultralow density polyethylene, linear low density polyethylene, high density polyethylene, high molecular weight polyethylene, ultrahigh molecular weight polyethylene), polypropylene (like oriented polypropylene, biaxially oriented polypropylene), cyclic olefin polymers (COP, like polynorbornene) or cyclic olefin copolymers (COC, like copolymers of ethylene and norbornene)].

In another form, the surface is at least partly made of hydrophilized polystyrene.

In one especially preferred form the surface is at least partly made of glass.

The term “surfaces is at least partly made of a material” usually means that at least 50%, preferably at least 80%, and in particular at least 95% of the surface is made of the material. The surface usually refers to that part of the surface of the biocompatible device which in general comes into contact with biological material (e.g. cells) under normal operating conditions. In a form the at least 50%, preferably at least 80%, and in particular at least 95% of the surface is made of glass.

The polymer P is a copolymer of at least one monomer M and at least one macromonomer (e.g. the ester E). In the context of this application, this shall mean that polymer P comprises these monomers in polymerized form. Preferably, the polymer P consists of the monomer M and the macromonomer, such as the ester E, and the cationic monomer C. In another form the polymer P is free of other monomers beside the monomer M and the macromonomer, such as the ester E, and the cationic monomer C.

In a preferred form the macromonomer is the ester E. The ester E is the ester of methacrylic acid and polyethylene oxide, and/or of acrylic acid and polyethylene oxide. In a preferred embodiment the polyethylene oxide is esterified on one end with (meth)acrylic acid and has been functionalized on the other end, for example by pro forma etherification with an alkyl group like methyl, ethyl, propyl or butyl, preferably methyl. The latter are normally obtained by alkoxylation of alcohols like methanol. In a preferred form the ester E is an ester of (meth)acrylic acid and polyethylene glycol mono C₁-C₄ alkyl ether, in particular an ester of (meth)acrylic acid and polyethylene glycol mono methyl ether.

In a less preferred embodiment said polyethylene oxide is esterified on one end with (meth)acrylic acid and bears a hydroxy group on the other end.

Polyethylene oxide in this context shall mean a polyalkylene oxide that consists essentially of oxyethylene units and optionally a terminal alkyl ether group. In particular, polyethylene oxide comprises less than 10 mol % of oxyalkylene units different from oxyethylene. Preferably, polyethylene oxide as used in this context comprises less than 5 mol %, more preferably less than 1 mol % of oxyalkylene units different from oxyethylene. In an especially preferred embodiment polyethylene oxide as used herein consists of oxyethylene units and a terminal alkyl ether group. Polyethylene oxide is in many cases prepared by ring opening polymerization of ethylene oxide using alcohols like methanol, ethanol, n/iso-propanol or n/sec/tert-butanol as a starter.

In one form the macromonomer is a polyethylene glycol (meth)acrylamide, which may be selected from mono-N or di-N substituted (meth)acrylamide, where mono-N substituted are preferred. The polyethylene glycol may be selected from the aforementioned polyethylene oxide.

Preferably, macromonomer (e.g. the ester E) has an average molar mass Mn of 300 to 10,000 g/mol, more preferably 500 to 10,000 and even more preferably 800 to 10,000 g/mol, especially preferably 1,000 to 10,000 g/mol and particularly preferably 1500 to 10,000 g/mol.

In another embodiment, the macromonomer (e.g. the ester E) has an average molar mass Mn of 300 to 8,000 g/mol, more preferably 300 to 5,000 and in particular 300 to 3,000 g/mol.

In especially preferred embodiments, macromonomer (e.g. the ester E) has an average molar mass Mn of 500 to 8000 g/mol, 1000 to 5000 g/mol, 800 to 3000 g/mol, 1000 to 3000 g/mol, 800 to 2500 g/mol.

The polymer P usually comprises 5-90 mol %, preferably 10-70 mol %, and in particular 20-mol % of the macromonomer, which may be selected from an ester E of (meth)acrylic acid and polyethylene oxide. The polymer P may comprise at least 1, 5, 10, 15, 20, 25, 30, 35 or 40 mol % of the macromonomer, which may be selected from an ester E of (meth)acrylic acid and polyethylene oxide. The polymer P may comprise up to 90, 80, 70, 60, 50, 40, or 30 mol % of the macromonomer, which is may be selected from an ester E of (meth)acrylic acid and polyethylene oxide.

The monomer M is selected from alkyl (meth)acrylate, aryloxyalkyl (meth)acrylate, alkyl (meth)acrylamide, or aryl (meth)acrylamide. Mixtures of monomer M are also possible. Preferably, monomer M is C₁-C₁₈ alkyl (meth)acrylate or phenoxyethyl (meth)acrylate. In one form monomer M is C₁-C₁₈ alkyl (meth)acrylate, preferably C₁-C₆ alkyl (meth)acrylate. In another form monomer M is phenoxyethyl (meth)acrylate.

Suitable alkyl (meth)acrylates are C₁-C₁₈ alkyl (meth)acrylate, preferably C₁-C₁₂ alkyl (meth)-acrylate, in particular C₁-C₆ alkyl (meth)acrylate. The alkyl unit of the alkyl (meth)acrylate may be linear, branched or cyclic, preferably linear or branched. In a preferred form alkyl (meth)acrylate is methyl acrylate, ethyl acrylate, propyl acrylate, butyl acrylate, butyl methacrylate, or methyl methacrylate. In particular the monomer M is selected from butyl acrylate and butyl methacrylate. In another form the monomer M is selected from n-butyl (meth)acrylate, preferably n-butyl methacrylate.

Suitable aryloxyalkyl (meth)acrylates are phenyloxyalkyl (meth)acrylates, such as phenoxyethyl acrylate and phenoxyethyl methacrylate, wherein phenoxyethyl acrylate is preferred.

Suitable alkyl (meth)acrylamides are N—C₁-C₂₂ alkyl (meth)acrylamides, preferably N—C₄-C₂₀ (meth)alkyl acrylamides, such as N-tert-butyl acrylamide, N-hexadecyl acrylamide, or N-octa-decyl acrylamide.

Suitable aryl (meth)acrylamides are N-aryl (meth)acrylamides, preferably N-phenyl acrylamide.

The polymer P usually comprises 15-75 mol %, preferably 20-70 mol %, and in particular 35-60 mol % of the monomer M, which may be a C₁-C₁₂ alkyl (meth)acrylate. The polymer P may comprise at least 10, 15, 20, 30, 35, 40 or 45 mol % of the monomer M, which may be a C₁-C₁₂ alkyl (meth)acrylate. The polymer P may comprise up to 90, 80, 70, 65, 60, 55, 50, 45, 40, 35 or mol % of the monomer M, which may be a C₁-C₁₂ alkyl (meth)acrylate.

The cationic monomer C may be selected from cationic ethylenically unsaturated N-containing monomers. Mixtures of different cationic monomers C are also possible.

The cationic monomer C usually forms ammonium salts with anions such as sulfate, C₁-C₄-alkyl sulfates and halides, in particular with chloride.

In one form the cationic monomer C can be vinyl- and allyl-substituted nitrogen heterocycles such as 2-vinylpyridine, 4-vinylpyridine, 2-allyl-pyridine, 4-allylpyridine, N-vinylimidazole, and 1-vinyl-3-methylimidazolium chloride.

In another form the cationic monomer C can be a monomer of formula (I)

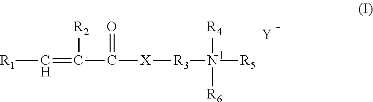

where

R₁ is H or C1-C4-alkyl, preferably H;

R₂ is H or methyl, preferably methyl;

R₃ is C1-C4-alkylene, preferably ethylene;

R₄, R₅ and R₆ are each independently H or C1-C30-alkyl, preferably H, methyl, ethyl, or propyl;

X is —O— or —NH—, preferably —O—; and

Y is Cl; Br; I; hydrogensulfate or methosulfate, preferably Cl⁻.

In a preferred form the cationic monomer C is selected from monomers of formula (I) where R₁ is H, and R₂ is H or methyl.

In another preferred form the cationic monomer C is selected from monomers of formula (I) where R₁ is H, and R₂ is H or methyl, R₃ is C2-C4-alkylene, and R₄, R₅ and R₆ are each methyl, ethyl, or propyl.

Preferred examples for the cationic monomer C are

Trimethylammoniumethyl acrylate chloride (TMAEC),

Trimethylammoniumethyl methacrylate chloride (TMAEMC),

2-(Dimethylamino)ethyl methacrylate,

tert-Butylaminoethyl methacrylate,

3-Acrylamidopropyl trimethylammonium chloride,

3-Methacrylamidopropyl trimethylammonium chloride,

N-[3-(Dimethylamino)propyl]methacrylamide,

N-[3-(Dimethylamino)propyl]acrylamide, and

1-Vinylimidazole.

The polymer P may comprise at least 1, 5, 10, 15, or 20 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

The polymer P may comprise up to 90, 80, 70, 60, 50, 40, or 30 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

The polymer P may comprise 5-60 mol %, preferably 10-45 mol %, and in particular 20-30 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

In a preferred form the polymer P comprises
5-90 mol % of the macromonomer, preferably 10-70 mol %, and in particular 15-35 mol %;
15-75 mol % of the monomer M, preferably 20-70 mol %, and in particular 35-60 mol %; and
5-60 mol % of the cationic monomer C, preferably 10-45 mol %, and in particular 15-35 mol %.

In a preferred form the polymer P comprises
5-90 mol % of the macromonomer, which may be selected from an ester E of (meth)acrylic acid and polyethylene oxide;
15-75 mol % of the monomer M, which may be a C₁-C₁₂ alkyl (meth)acrylate; and
5-60 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

The molar amounts of the monomers in the polymer P usually sums up to 100 mol %.

In another preferred form the polymer P comprises
10-70 mol % of the macromonomer, which may be selected from an ester E of (meth)acrylic acid and polyethylene oxide;
20-70 mol % of the monomer M, which may be a C₁-C₁₂ alkyl (meth)acrylate; and
10-45 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

In another preferred form the polymer P comprises
15-35 mol % of the macromonomer, which may be selected from an ester E of (meth)acrylic acid and polyethylene oxide;
35-60 mol % of the monomer M, which may be a C₁-C₁₂ alkyl (meth)acrylate; and
15-35 mol % of the cationic monomer C, which may be selected from monomers of formula (I) where R₁ is H, and R₂ is H or methyl, R₃ is C2-C4-alkylene, and R₄, R₅ and R₆ are each methyl, ethyl, or propyl.

The polymer P has usually a number average molar mass Mn of 2,000 to 100,000 g/mol, preferably of 3,000 to 80,000, and in particular of 5,000 to 40,000. The polymer P has usually a weight average molar mass Mw of 3,000 to 200,000 g/mol, preferably of 4,000 to 100,000, and in particular of 5,000 to 80,000. All values for the average molar mass Mn or Mw given in this application may be determined by gel permeation chromatography (GPC), e.g. using the method as described in the experimental section of this application.

The polymer P is preferably a statistical copolymer in which monomer M, the macromonomer (e.g. the ester E), and the cationic monomer C are distributed statistically.

The polymer P is normally prepared by radical polymerization of the monomer M, the macromonomer (e.g. the ester E) and the cationic monomer C, e.g. by solution polymerization or emulsion polymerization.

Preferably the polymer P is prepared by solution polymerization. “Solution polymerization” means that all starting materials are at least partly dissolved in the same solvent and that the polymerization reaction takes place in homogenous phase, without additional surfactants having to be present. In one preferred embodiment, the monomer M and the macromonomer (e.g. the ester E) are dissolved in suitable solvents like alcohols like methanol, ethanol, 1-propanol, 2-propanol, butanol or mixtures thereof and are then polymerized. Preferably, such solvents for the solution polymerization comprise at least 50% by weight, preferably 70% and more preferably 80% by weight of alcohols like methanol, ethanol, 1-propanol, 2-propanol, butanol or mixtures thereof. Preferably, such solvents for the solution polymerization comprise 20% by weight or less, preferably 10% by weight or less of water.

The radical polymerization can be initiated by oxidative radical starters like organic peroxides (e.g. tert-butyl-2,2-dimethylpropaneperoxoate, sodium persulfate, potassium persulfate, metachloroperbenzoic acid) or azo starters like azo-bisisobutyrodinitrile or 2,2′-Azobis(2-methyl-butyronitrile).

The biocompatible device comprises on its surface an adsorbed layer of a polymer P, where the adsorbed layer may be a self-assembled monolayer of the polymer P.

A “self-assembled monolayer” means typically a molecular assembly formed spontaneously on a surface by adsorption. A self-assembled monolayer forms usually spontaneously on such surfaces without any further process step being required. Self-assembled monolayers can for example be characterized by atomic force microscopy (AFM) or X-ray photoelectron spectroscopy (XPS) or in situ methods such as quartz crystal microbalance or surface plasmon resonance spectroscopy.

The self-assembled monolayer of the polymer P normally has a thickness that correlates with the size of the individual molecules adsorbed to that surface, e.g. it is normally smaller than 100 nm, such as 1 to 50 nm, preferably 1 to 20 nm, and in particular 1 to 10 nm.

In a form the biocompatible device comprises on its surface an adsorbed layer of a polymer P which is a copolymer of at least one macromonomer selected from an ester E of (meth)acrylic acid and polyethylene oxide, and at least one monomer M selected from alkyl (meth)acrylate or aryloxyalkyl (meth)acrylate, and at least one cationic monomer C where the biocompatible device is a device for cultivating cells, and where the surface is at least partly made of glass.

In a form the biocompatible device comprises on its surface an adsorbed layer of a polymer P which is a copolymer of at least one macromonomer selected from an ester E of (meth)acrylic acid and polyethylene oxide, and at least one monomer M selected from butyl (meth)acrylate (e.g. n-butyl methacrylate), and at least one cationic monomer C where the biocompatible device is a device for cultivating cells, and where the surface is at least partly made of glass.

In another form the biocompatible device comprises on its surface an adsorbed layer of a polymer P which is a copolymer of at least one macromonomer selected from an ester E of (meth)acrylic acid and polyethylene oxide, and at least one monomer M selected from C₁-C₁₈ alkyl (meth)acrylate or phenoxyethyl (meth)acrylate, and the cationic monomer C where the biocompatible device is a device for cultivating cells, and where the surface is at least partly made of glass.

In another form the biocompatible device comprises on its surface an adsorbed layer of a polymer P which is a copolymer of at least one macromonomer selected from an ester E of (meth)acrylic acid and polyethylene oxide, and at least one monomer M selected from butyl (meth)acrylate (e.g. n-butyl methacrylate), and the cationic monomer C where the biocompatible device is a device for cultivating cells, and where the surface is at least partly made of glass.

In another form the biocompatible device comprises on its surface an adsorbed layer of a polymer P which is a copolymer of at least one macromonomer selected from an ester E of (meth)acrylic acid and polyethylene oxide, and at least one monomer M selected from C₁-C₁₈ alkyl (meth)acrylate or phenoxyethyl (meth)acrylate, and the cationic monomer C
where the polymer P comprises
5-90 mol % of the macromonomer, preferably 10-70 mol %, and in particular 15-35 mol %;
15-75 mol % of the monomer M, preferably 20-70 mol %, and in particular 35-60 mol %; and
5-60 mol % of the cationic monomer C, preferably 10-45 mol %, and in particular 15-35 mol %
where the biocompatible device is a device for cultivating cells, and
where the surface is at least partly made of glass.

The invention also relates to a process for making the biocompatible device comprising the following steps:
A) providing a biocompatible device, and
B) applying to the surface of the biocompatible device a solution S of the polymer in a solvent L.

In another form the invention relates to a process for making the biocompatible device which comprises on its surface an adsorbed layer of a polymer P which is a copolymer of at least one macromonomer selected from an ester E of (meth)acrylic acid and polyethylene oxide or a polyethylene glycol (meth)acrylamide, and at least one monomer M selected from alkyl (meth)acrylate, aryloxyalkyl (meth)acrylate, alkyl (meth)acrylamide or aryl (meth)acrylamide, and at least one cationic monomer C comprising the following steps:
A) providing a biocompatible device, and
B) applying to the surface of the biocompatible device a solution S of the polymer P in a solvent L.

In another form the invention relates to a process for making the biocompatible device which comprises on its surface an adsorbed layer of a polymer P which is a copolymer of at least one macromonomer selected from an ester E of (meth)acrylic acid and polyethylene oxide or a polyethylene glycol (meth)acrylamide, and at least one monomer M selected from butyl (meth)acrylate (e.g. n-butyl methacrylate), and at least one cationic monomer C comprising the following steps:
A) providing a biocompatible device, and
B) applying to the surface of the biocompatible device a solution S of the polymer P in a solvent L.

Typically, the biocompatible device is obtainable by the process for making the biocompatible device.

The invention also relates to the solution S comprising the polymer P in the solvent L, where the solvent L comprises an alcohol, such as methanol, ethanol, n/iso-propanol or n/sec/iso/tert-butanol. Preferably, the solution S comprises 0.01 to 5 wt % of the polymer P and 5 to 90 wt % of the solvent L.

The solution S normally comprises 0.001 to 10% by weight of polymer P based on the solution S, preferably 0.05 to 5% by weight and even more preferably 0.05 to 0.3% by weight.

The solution S may comprise further additives in addition to the polymer P and the solvent L. Suitable further additives are polyethylene oxide (such as the polyethylene oxide described above in the ester E), block polymers (such as di- or tri-block polymers) of polyethylene oxide and polypropylene oxide (such as the triblock polymer polyethylene oxide-polypropylene oxide-polyethylene oxide), or mixtures thereof. The solution S may comprise 0.001 to 10% by weight, preferably 0.05 to 5% by weight of the further additives.

Suitable solvents L are water or alcohols like methanol, ethanol, n/iso-propanol or n/sec/iso/tert-butanol. Preferably, the solvent L comprises water and alcohol. Suitable alcohols are C₁ to C₁₂ alcohols, preferably C₁ to C₈ alcohols, and in particular C₁ to C₆ alcohols. In another form the solvent L comprises an alcohol, such as methanol, ethanol, n/iso-propanol or n/sec/iso/tert-butanol, preferably ethanol or n/iso-propanol. In another form the solvent L is a mixture of water and an alcohol, preferably a mixture of water and ethanol or a mixture of water and isopropanol.

Preferably, the solution S is an aqueous solution. “Aqueous” in this context shall mean that said polymer P is dissolved in a solvent or solvent mixture that comprises at least 50% by weight, preferably at least 70% by weight, more preferably at least 90% by weight and particularly preferably at least 99% by weight of water. In a preferred embodiment, the solvent L in which said at least one polymer P is dissolved is an aqueous solution. The aqueous solution S is usually a clear solution without any turbidity. In another embodiment, the aqueous solution S comprises polymer P at least partly in dissolved state but shows turbidity. Preferably, solution S is an aqueous solution comprising at least 50% of water.

In another preferred embodiment, solution S comprises at least 5% by weight, preferably at least 10% by weight, more preferably at least 90% by weight and particularly preferably at least 15% by weight of at least one alcohol like methanol, ethanol, n/iso-propanol or n/sec/iso/tert-butanol.

In another preferred embodiment, solution S comprises at least 50% by weight, preferably at least 70% by weight, more preferably at least 90% by weight and particularly preferably at least 99% by weight of at least one alcohol like methanol, ethanol, n/iso-propanol or n/sec/iso/tert-butanol.

In another preferred embodiment, the solvent L comprises at least 50% by weight, preferably at least 55% by weight, and in particular at least 60% by weight of at least one alcohol like methanol, ethanol, n/iso-propanol or n/sec/iso/tert-butanol.

In another preferred form, the solvent L comprises ethanol and water, and the ethanol content may be at least 1 wt % (e.g. at least 10 wt %), preferably from 1 to 99 wt %, or 5 to 90 wt %, or 10 to 80 wt %, or 10 to 50 wt %, or 13 to 30 wt %.

In another preferred form, the solvent L comprises isopropanol and water, and the isopropanol content may be at least 5 wt % (e.g. at least 10 wt %), preferably from 1 to 99 wt %, or 5 to 90 wt %.

In another preferred form the solvent L comprises ethanol and water, and the ethanol content may be at least 5 wt %, or the solvent L comprises isopropanol and water, and the isopropanol content may be at least 5 wt %.

In another form the solution S comprises 0.001 to 10% by weight of polymer P based on the solution (preferably 0.05 to 5% by weight and even more preferably 0.05 to 0.3% by weight), and a) the solvent L comprises ethanol and water, and the ethanol content may be at least 5 wt %, or b) the solvent L comprises isopropanol and water, and the isopropanol content may be at least 5 wt %.

In one form the process for making the biocompatible device comprises the following steps: A) providing the biocompatible device, and B) applying to the surface of the biocompatible device a solution S of the polymer in a solvent L, where the solvent L comprises at least 5% by weight of at least one alcohol like methanol, ethanol, n/iso-propanol or n/sec/iso/tert-butanol.

In one form the process for making the biocompatible device comprises the following steps: A) providing the biocompatible device, and B) applying to the surface of the biocompatible device a solution S of the polymer in a solvent L, where the solvent L comprises ethanol and water, and the ethanol content may be at least 5 wt %, or the solvent L comprises isopropanol and water, and the isopropanol content may be at least 5 wt %.

In another form the process for making the biocompatible device comprises the following steps: A) providing the biocompatible device, and B) applying to the surface of the biocompatible device a solution S of the polymer in a solvent L, where the solution S comprises 0.001 to 10% by weight of polymer P based on the solution S (preferably 0.05 to 5% by weight and even more preferably 0.05 to 0.3% by weight), and a) the solvent L comprises ethanol, and the ethanol content may be at least 5 wt %, or b) the solvent L comprises ethanol and water, and the ethanol content may be at least 5 wt %.

Typically, the biocompatible device in step A) corresponds to the biocompatible device before it was treated by step B).

Optionally, the process may further comprise the following step:
C1) removing the supernatant solution S; or
C2) removing the solvent L.

In one form the process may further comprise the following step:
C1) removing the supernatant solution S.

Upon the application of solution S to the surface, the polymer P will normally self-organize to form a layer, in many cases a self-assembled monolayer, of polymer P on the surface. In many cases it will be sufficient to apply solution S to surface 0 and wait for a short period of time, for example 1 second to 1 day, preferably 1 second to 4 hours, or 1 second to 30 min, or 1 second to 10 min, or 1 second to 5 min. Next, the supernatant solution S can be removed, for example mechanically (for example by wiping or using a pipette) or by exchanging solution S by water or an (aqueous) solution that does not comprise of polymer P.

The step C1) of removing the supernatant solution S may comprise exchanging solution S for a solution or for a pure solvent or solvent mixture that does not contain polymer P (for example a cell culture medium or water or a buffer solution).

The step C1) usually results in the formation of self-adsorbed monolayer of the polymer P on the surface.

In another form the process may further comprise the following step:
C2) removing the solvent L.

The removing of the solvent L may be achieved by drying or evaporation of the solvent L, e.g. at ambient or elevated temperature, or at ambient or reduced pressure. Preferably, the removing of the solvent L is achieved by drying at ambient temperature and ambient pressure.

Step C2) may be considered as a forced deposition wherein polymer P is applied from a solution and subsequently the solution is not withdrawn as a whole, but only the solvent L is removed, for example by evaporation, leaving the formerly dissolved polymer P deposited on the surface. A forced deposition can be applied by filling wells of a plate with the solution of polymer P followed by drying, alternatively, a forced deposition can be applied by dip coating, spin-coating, spraying, draw-down bar application, and other methods.

The thickness and the amount per area of the adsorbed layer usually depends on the process for making the biocompatible device, especially if step C1) or C2) were applied. In general, the adsorbed layer may have a thickness of 1 nm to 10 μm. In general, the polymer P is adsorbed on the surface in an amount of at least 50 ng/cm², preferably at least 100 ng/cm², which may be determined by quartz crystal microbalance.

When the biocompatible device was obtained by the process comprising step C1) of removing the supernatant solution S, then the adsorbed layer may have a thickness of 1 nm to 100 nm, preferably 1 to 20 nm, and in particular 1 to 10 nm.

When the biocompatible device was obtained by the process comprising step C1) of removing the supernatant solution S, then the polymer P is adsorbed in an amount of 50 to 5000 ng/cm², preferably 100 to 3000 ng/cm², and in particular 200 to 1000 ng/cm² on the surface.

When the biocompatible device was obtained by the process comprising step C2) of removing the solvent L, then the adsorbed layer may have a thickness of 0.01 μm to 100 μm, preferably 0.1 to 20 μm, and in particular 0.5 to 10 μm.

When the biocompatible device was obtained by the process comprising step C2) of removing the solvent L, then the polymer P is adsorbed in an amount of 0.5 to 5000 μg/cm², preferably 5 to 500 μg/cm², and in particular 30 to 300 μg/cm² on the surface.

The process for making the biocompatible device may be achieved in situ when cultivating cells in the supernatant medium above the surface of the biocompatible device. The process for making the biocompatible device may comprise the following steps:
A) providing the biocompatible device (e.g. a device for cultivating cells),
B) applying to the surface of the biocompatible device a solution S of the polymer P in a solvent L, where the solution S is a cell culture medium, and
C) cultivating cells in the supernatant medium above the surface of the biocompatible device.

The cell culture medium may comprise 0.001 to 30% by weight, preferably 0.01 to 5% by weight and even more preferably 0.05 to 1% by weight of the polymer P based on the medium.

It is assumed that through this process adsorbed layer of the polymer P is prepared in situ that allows for efficient cultivation of such cell. Suitable solvent L are described above.

Any kind of cell culture medium may be used, wherein aqueous cell culture mediums with the solvent L comprising water are preferred. The cell culture medium may contain cells, e.g. those which are cultivated in step C). The supernatant medium of step C) usually corresponds to the solution S which is a cell culture medium. The supernatant medium of step C) usually comprises the polymer P and the solvent L (preferably water) applied in step B).

Between the step B) of applying the solution S and the step C) of cultivating the cells is preferably no removing of the solution S or the solvent L. Usually, the amount of the solution S in the biocompatible device in step C) is at least the same as in step B). Usually, the solution S is identical in step B) and in step C). Usually, the supernatant medium in step C) is identical to the solution S of step B).

The present invention also relates to a process for cultivating cells, comprising the following steps:
a) providing the biocompatible device which is a device for cultivating cells, and
b) cultivating cells in the supernatant medium above the surface of the biocompatible device.

Optionally, the biocompatible device is subjected to a sterilization prior to cultivating cells in step b), e.g. by exposing the biocompatible device to gaseous ethylene oxide, electron-beam, x-ray or gamma-irradiation. In case the biocompatible device is subjected to sterilization, the biocompatible device is obtainable by the process comprising the step C2) of removing the solvent L.

The cells may be adherent cells or non-adherent cells, where adherent cells are preferred. Suitable cells are any cells derived from multicellular organisms, preferably cells derived from plant, animal (such as simian, rat, mouse, hamster, dog, chicken, fish (preferably zebra fish) or insect origin) or human, in particular from human, simian, rat, mouse, hamster, dog, chicken, fish (preferably zebra fish) or insect origin.

Preferred cells are
stem cells,
primary cells,
progenitor cells,
differentiated cells,
cancer cells, and
cancer cell derived cell lines.

More preferred cells are stem cells and cancer cells.

Examples for stem cells are induced pluripotent stem cells, adult stem cells or embryonic stem cells. Examples for adult stem cells are hematopoietic stem cells, mammalian stem cells, intestinal stem cells, mesenchymal stem cells, endothelial stem cells, neural stem cells, olfactory adult stem cells, neural crest stem cells, or testicular cells, where mesenchymal stem cells and hematopoietic stem cells are preferred.

Examples for primary cells are cells derived from human (e.g. cells human-derived hepatocytes) or animal organs (e.g. organs like mammalian gland, colon, liver, kidney, pancreas).

Examples for differentiated cells are blood cells (e.g. human blood cells) and cells derived from human or animal organs (e.g. organs like mammalian gland, colon, liver, kidney, pancreas, prostate, lung, stomach or brain).

Examples for cancer cells are human cancer cells (e.g. breast cancer cells, prostate cancer cells, or HeLa cells), or immortalized cells.

In a form the process for cultivating cells comprises the following steps:
a) providing the biocompatible device which is a device for cultivating cells, and
b) cultivating cells in the supernatant medium above the surface of the biocompatible device,
where the biocompatible device comprises on its surface an adsorbed layer of a polymer P which is a copolymer of at least one macromonomer selected from an ester E of (meth)acrylic acid and polyethylene oxide, and at least one monomer M selected from alkyl (meth)acrylate or aryloxyalkyl (meth)acrylate, and at least one cationic monomer C, and
where the surface is at least partly made of glass.

In a form the process for cultivating cells comprises the following steps:
a) providing the biocompatible device which is a device for cultivating cells, and
b) cultivating cells in the supernatant medium above the surface of the biocompatible device,
where the biocompatible device comprises on its surface an adsorbed layer of a polymer P which is a copolymer of at least one macromonomer selected from an ester E of (meth)acrylic acid and polyethylene oxide, and at least one monomer M selected from alkyl (meth)acrylate or aryloxyalkyl (meth)acrylate, and at least one cationic monomer C,
where the surface is at least partly made of glass.

In a form the process for cultivating cells comprises the following steps:
a) providing the biocompatible device which is a device for cultivating cells, and
b) cultivating cells in the supernatant medium above the surface of the biocompatible device,
where the biocompatible device comprises on its surface an adsorbed layer of a polymer P which is a copolymer of at least one macromonomer selected from an ester E of (meth)acrylic acid and polyethylene oxide, and at least one monomer M selected from butyl (meth)acrylate (e.g. n-butyl methacrylate), and at least one cationic monomer C, and
where the surface is at least partly made of glass.

The invention has various advantages: The biocompatible devices are suitable for cultivating cell cultures, especially for the cultivation of normally adherent cells as non-adherent cell cultures, where they show improved anti-adhesive behavior. Avoiding cell adhesion in this device improves three-dimensional growth of cells, which resembles more closely the natural growth of cells by increasing cell to cell interaction and resembling natural drug, oxygen and nutrient gradients. They are easy and economical to make, they allow for the growing of cell cultures with a high circularity (meaning a round cell morphology; the circularity is a parameter defining how spherical an object is; it is calculated by 4*PI* cell area/square of cell perimeter), they allow for the growing of cells with a very low degree of adhesion to the surface, they allow for the growing of adherent cells with a round spheroid morphology, they allow for the growing of cells that are essentially free floating in the medium and can be easily harvested. Culturing in biocompatible devices allows the generation of three-dimensional cell aggregates of consistent size and shape. The usage of a single polymer layer, in contrast to thick surface coatings, allows for a surface modification which is uniform and does not influence the geometry of the biocompatible device.

### EXAMPLES

Molecular weights were determined by Size Exclusion Chromatography using a mixed bed scouting column for water soluble linear polymers at 35° C. The eluent used was 0.01 M phosphate buffer at pH=7.4 containing 0.01 M sodium azide. The polymer used as 1.5 mg/mL concentrated solution in the eluent. Before injection all samples were filtered through a 0.2 μm filter. The calibration was carried out with narrow poly(ethylene glycol) samples having molecular weights between 106 and 1,378,000 g/mol.

### Example 1: Polymer Preparation

32.3 parts by weight of methoxy polyethylene glycol methacrylate (50% aqueous solution, molecular weight (av.) 2100 g/mol) dissolved in 12.7 parts by weight of isopropanol and 2 parts by weight of 2-Trimethylammoniumethyl methacrylate chloride (TMAEMC, 80% aqueous solution) and 2.2 parts by weight of n-butyl methacrylate dissolved in 5.8 parts by weight of isopropanol were added to 400 parts by weight of isopropanol. The reaction mixture was stirred at 125 rpm under nitrogen and heated to 80° C. Simultaneously, 291.2 parts by weight of the methoxy polyethylene glycol methacrylate dissolved in 113.8 parts by weight of isopropanol were added within 3 h, 18.2 parts by weight of TMAEMC (80% aqueous solution) and 19.9 parts by weight of n-butyl methacrylate dissolved in 51.9 parts by weight of isopropanol were added within 2 h and 2.7 parts by weight of tert-butyl peroxypivalate (75% solution) in 47.3 parts by weight of isopropanol were added within 4 h. Subsequently, 2.7 parts by weight of tert-butyl peroxypivalate (75% solution) in 17.3 parts by weight of isopropanol were added within 1 h. Afterwards, the reaction was kept for 2 hours at 80° C. Subsequently, the reaction was cooled down to the ambient temperature and subjected to purification by water steam distillation. The molar ratio of methoxy polyethylene glycol methacrylate:TMAEMC:n-butyl methacrylate was of 1:1:2. The resulting polymer had a Mn 6500 g/mol and about Mw 9000 g/mol.

### Example 2: Polymer Preparation

The polymer preparation of Example 1 was repeated with the same monomers, but a different molar ratio of methoxy polyethylene glycol methacrylate:TMAEMC:n-butyl methacrylate of 2:1:1.

### Example 3: Coating of Model Surfaces and Anti-Adhesive Evaluation

Method: The amounts of polymer P or milk proteins absorbed on the surface were determined by Quartz-Crystal Microbalance (QCM), which measures the resonance frequency of a freely oscillating quartz sensor after excitation in contact with the liquid medium. QCM measurements were performed using standard flow-through setups with a flow rate of 50 μL/min at 23° C. A measured shift in resonance frequency scales inversely with mass changes at the sensor surface. The adsorbed amounts were calculated from the shift of the 7th overtone of the resonance frequency according to the method of Sauerbrey. The mass sensitivity or mass changes is estimated to ˜10 ng/cm² in all experiments. Measurements were performed on a quartz sensor quartz coated with silicon dioxide, soda lime glass, or with borosilicate glass.

Adsorption of Polymer P and of Milk Proteins:

The experiments comprised the following steps and used an aqueous HEPES (4-(2-Hydroxyethyl)-1-piperazine ethanesulfonic acid) buffer (10 mmol/L) with pH 7 (“buffer”):
1) buffer until a stable baseline was achieved;
2) 2 h 0.1 wt % polymer P solution in buffer;
3) 2 h buffer;
4) 0.5 h 0.1 wt % milk powder in buffer;
5) 0.5 h buffer.

Milk fouling was monitored during exposure of the samples to 0.1 wt % solutions of milk powder for 0.5 h. The final mass change was recorded after another 0.5 h of rinsing with buffer (steps 4) and 5) above). The results (based on double determination) are given in Table 1-3.

TABLE 1
Silicon dioxide surface

| | Amount of | Milk Protein | Milk Protein |
|---|---|---|---|
| | polymer P | Adhesion | Adhesion |
| Polmyer P | [ng/cm²] | [ng/cm²] | [wt %] |
| No polymer (comparative) | — | 364 | 100 |
| Ex. 1 | 401 | 53 | 14.6 |
| Ex. 2 | 315 | 41 | 11.3 |

TABLE 2
on soda lime glass surface

| | Amount of | Milk Protein | Milk Protein |
|---|---|---|---|
| | polymer P | Adhesion | Adhesion |
| Polmyer P | [ng/cm²] | [ng/cm²] | [wt %] |
| No polymer (comparative) | — | 457.5 | 100 |
| Ex. 1 | 436 | 72 | 15.7 |
| Ex. 2 | 442 | 54 | 11.8 |

TABLE 3
on borosilicate glass surface

| | Amount of | Milk Protein | Milk Protein |
|---|---|---|---|
| | polymer P | Adhesion | Adhesion |
| Polmyer P | [ng/cm²] | [ng/cm²] | [wt %] |
| No polymer (comparative) | — | 697 | 100 |
| Ex. 1 | 543 | 33 | 4.7 |
| Ex. 2 | 532 | −34 | −4.9 |

### Example 4: Evaluation of Cell Adherence in Cell Culture

The adsorbed layer of polymer P on the glass plates have been made as follows. As basis plate a SiO₂ coated plate was used (96 PLATE+GL CT 7 MM RD FLAT BASE, Greiner: CRMA60180-P304).

Solid polymer P powder was dissolved in 20% ethanol (v/v) and 80% Milli-Q-water at a final concentration of 1% and passed through a filter for sterilization. Sterile solution was pipetted into wells (200 μl per well) and incubated at room temperature for 3 min. After incubation the polymer solution was removed using a standard pipette and dried for 30 min, followed by cell seeding (HUVEC, 6000 cell/200 μl/well) as described previously.

The adherence was characterized using an automated light microscopy (IncuCyte System, Sartorius) with a 4× objective, phase contrast and photo documentation taken whole well picture per well. Quantitative analysis of cell adherence was performed by means of automatic microscopy and image analysis using the corresponding software of the automated microscope (IncuCyte S3 2018B, confluence mask). The percentage of adherent cells was determined after 2 days of culture via surface analysis within the well. Cells that do not adhere to the surface of the plate, accumulate into cell aggregates. In contrast, adherent cells spread on the surface of the cell culture plate can be quantified via a confluence analysis, which determines the surface covered by cells. The results were summarized in Table 4.

The results demonstrate that the Polymer P reduces the deposition of cells on glass surfaces.

TABLE 4

| Polymer P | % Area covered by adherent cells |
|---|---|
| no polymer (comparative) | 78 ± 1.7 |
| Ex 1 | 10 ± 0.9 |

1. A biocompatible device which comprises on its surface an adsorbed layer of a polymer P which is a copolymer of
at least one macromonomer selected from an ester E of (meth)acrylic acid and polyethylene oxide or a polyethylene glycol (meth)acrylamide,
at least one monomer M selected from alkyl (meth)acrylate, aryloxyalkyl (meth)acrylate, alkyl (meth)acrylamide or aryl (meth)acrylamide, and
at least one cationic monomer C selected from cationic ethylenically unsaturated N-containing monomers.

2. The biocompatible device according to claim 1, where the biocompatible device is a biosensor or a device for cultivating cells.

3. The biocompatible device according to claim 2, where the device for cultivating cells is selected from cell culture flasks, cell culture dishes, cell culture plates, cell culture bags, reactors, tubes, probes, vials, bottles, pipettes, pipette tips, syringes, microscopic slides, microfluidic devices, glass capillaries, biochips, SiO2 wavers or SiO2 arrays.

4. The biocompatible device according to claim 1, where the macromonomer is the ester E of (meth)acrylic acid and polyethylene oxide.

5. The biocompatible device according to claim 1, where the monomer M is C₁-C₆ alkyl (meth)acrylate.

6. The biocompatible device according to claim 1, where the cationic monomer C is a monomer of formula (I) 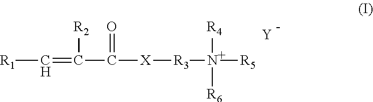
where
R₁ is H or C1-C4-alkyl;
R₂ is H or methyl;
R₃ is C1-C4-alkylene;
R₄, R₅ and R₆ are each independently H or C1-C30-alkyl;
X is —O— or —NH—; and
Y is Cl; Br; I; hydrogensulfate or methosulfate.

7. The biocompatible device according to claim 1, where the polymer P comprises
5-90 mol % of the macromonomer;
15-75 mol % of the monomer M; and
5-60 mol % of the cationic monomer C,
and where the molar amounts of the monomers sums up to 100%.

8. The biocompatible device according to claim 1, where the surface is free of other layers beside the adsorbed layer of the polymer P.

9. The biocompatible device according to claim 1, where the surface is at least partly made of glass.

10. The biocompatible device according to claim 1, where there are no covalent chemical bonds between the polymer P and the surface.

11. A process for making a biocompatible device which comprises on its surface an adsorbed layer of a polymer P which is a copolymer of
at least one macromonomer selected from an ester E of (meth)acrylic acid and polyethylene oxide or an polyethylene glycol (meth)acrylamide,
at least one monomer M selected from alkyl (meth)acrylate, aryloxyalkyl (meth)acrylate, alkyl (meth)acrylamide or aryl (meth)acrylamide, and
at least one cationic monomer C selected from cationic ethylenically unsaturated N-containing monomers,
comprising the following steps:
A) providing a biocompatible device, and
B) applying to the surface of the biocompatible device a solution S of the polymer P in a solvent L.

12. The process according to claim 11, further comprising the following step:
C1) removing the supernatant solution S; or
C2) removing the solvent L.

13. The process according to claim 11, where the solvent L comprises an alcohol.

14. A solution S comprising the polymer P as defined in claim 1 in a solvent L, where the solvent L comprises an alcohol.

15. The solution S according to claim 14 comprising 0.01 to 5 wt % of the polymer P and 5 to 90 wt % of the alcohol.

16. A process for cultivating cells, comprising the following steps:
a) providing the biocompatible device which is a device for cultivating cells as defined in claim 1, and
b) cultivating the cells in the supernatant medium above the surface of the biocompatible device.
